(19) United States
(12) Patent Application Publication
(10) Pub. No.: US 2024/0254469 A1
(43) Pub. Date: Aug. 1, 2024

(54) Hybrid Antimicrobial Protein Having Strong Bactericidal Effect and Application thereof

(71) Applicant: Jiangxi Yuansheng Biological Technology Co., Ltd., Yichun (CN)

(72) Inventors: Hairong LU, Shanghai (CN); Qingshan HUANG, Shanghai (CN); Guodong LI, Shanghai (CN); Jinjiang HUANG, Shanghai (CN); Xiaowei ZHAO, Shanghai (CN); Wanying LU, Shanghai (CN)

(73) Assignee: Jiangxi Yuansheng Biological Technology Co., Ltd., Yichun (CN)

(21) Appl. No.: 17/768,846

(22) PCT Filed: Sep. 22, 2020
(86) PCT No.: PCT/CN2020/116756

§ 371 Date: Mar. 22, 2024

(30) Foreign Application Priority Data
Oct. 14, 2019 (CN) 201910975220.9

(51) Int. Cl. C12N 9/36; A61K 38/00; A61P 31/04

(52) U.S. Cl. CPC C12N 9/2462; A61P 31/04; A61K 38/00; C07K 2319/00

(57) ABSTRACT

The present invention provides a hybrid antimicrobial protein having a strong bactericidal effect and an application thereof. Said hybrid antimicrobial protein having a strong bactericidal effect is a hybrid protein with the amino acid sequence of SEQ ID NO: 1 which named AB469; or a hybrid protein including a sequence similar to a catalytic domain of the AB469, or a hybrid protein including a sequence similar to a binding domain of the AB469; or, a hybrid protein including the catalytic and binding domains with positions interchanged of the AB469.

### INCORPORATION BY REFERENCE OF SEQUENCE LISTING ON A SCII TEXT FILE

The content of the following submission on ASCII text file is incorporated herein by reference in its entirety: a computer readable form (CRF) of the Sequence Listing (file name: 210816_sequence_listing_ST25.txt, date recorded: Mar. 21, 2024, size: 21 KB).

### TECHNICAL FIELD

The present invention relates to a field of hybrid antimicrobial protein.

### BACKGROUND OF THE INVENTION

Antibiotics have been widely used in the fields of medical and health services and agricultural breeding, and have played an important role in treatment of infectious diseases and protection of public health security. However, with extensive use of the antibiotics, bacteria have become increasingly resistant to the antibiotics. It resulted clinical treatment difficulties and increased mortality in the world and therefor seriously threatened human health.

Multi-drug resistant bacteria mainly include Acinetobacter baumannii, Pseudomonas aeruginosa, Enterobacteriaceae, Enterococcus and Staphylococcus aureus, in which Gram-negative bacteria (G⁻ bacteria) have a largest proportion, and particularly drug resistance of the Acinetobacter baumannii has become more and more serious in recent years (Mezzatesta M L, D' Andrea M Mezza Testa, Migliavacca R, et al. Epidemiological characterization and distribution of carbapenem-resistant Acinetobacter baumannii clinical isolates in Italy [J]. Clinical Microbiology and Infection, 2012, 18(2): 160-166. Durante-Mangoni E, Zarrilli R. Global spread of drug-resistant Acinetobacter baumannii: molecular epidemiology and management of antimicrobial resistance [J]. Future microbiology, 2011, 6(4): 407-422.). Among 12 deadliest drug-resistant bacteria ranked by the World Health Organization (WHO), the Acinetobacter baumannii, Pseudomonas aeruginosa and Enterobacteriaceae which resistant to carbapenem antibiotics are the most serious. It is urgent to develop new antimicrobial drugs, especially antimicrobial drugs with novel action modes different from the traditional antibiotics.

The traditional antibiotics have broad-spectrum antimicrobial properties and take the same effect on normal microbial flora. During extensive use of the antibiotics, the normal microbial flora also envolved drug resistance. As drug-resistant genes are transmitted between the normal microbial flora and pathogenic bacteria, the probability of drug-resistant bacteria is significantly increased. As a main component of a bacterial cell wall, peptidoglycan is a three-dimensional network structure formed by cross-linking a sugar chain, a peptide chain and a peptide bond bridge. The peptidoglycan is used for maintaining the mechanical strength of the cell wall and preventing bacteria from being affected by external factors. As a class of proteins that can specifically lyse the peptidoglycan of the bacterial cell wall, peptidoglycan hydrolases mainly include bacteriophage lysins, bacteriocins and bacterial autolysins (Vollmer W, Joris B, Charlier P, et al. Bacterial peptidoglycan(murein) hydrolases [J]. FEMS microbiology reviews, 2008, 32(2): 259-286.). For example, a Staphylococcus aureus bacteriophage lysin LysK, a Staphylococcus simulans bacteriocin lysostaphin and a Streptococcus pneumoniae autolysin LytA are included. A peptidoglycan hydrolase generally consists of a catalytic structural domain (catalytic domain) used for hydrolyzing the peptidoglycan and a targeting structural domain (binding domain) used for specifically targeting a bacterial cell wall.

When being added into a bacterial solution, the peptidoglycan hydrolase can specifically bind to sensitive bacteria and rapidly lyse cell walls. It has been extensively proved that the peptidoglycan hydrolase has antimicrobial activity in vivo and in vitro (Gerstmans H, Rodriguez-Rubio L, Lavigne R, et al. From endolysins to Artilisin®s: novel enzyme-based approaches to kill drug-resistant bacteria [J]. Biochemical Society Transactions, 2016, 44(1): 123-128. Guo M, Feng C, Ren J, et al. A Novel Antimicrobial Endolysin, LysPA26, against Pseudomonas aeruginosa [J]. Frontiers in Microbiology, 2017, 8:293.). The peptidoglycan hydrolase can quickly kill specific bacteria by directly lysing the peptidoglycan of the bacteria, has a narrow antimicrobial spectrum and a bactericidal mechanism different from the traditional antibiotics, and can be used repeatedly for a long time without easy induction drug resistance. The peptidoglycan hydrolase can directly lyse the bacteria in every growth cycle. By using the narrow-spectrum peptidoglycan hydrolase, the normal microbial flora is hardly affected.

However, there are some problems need to be improved when natural peptidoglycan hydrolases are directly used, for example, low antimicrobial activity, very narrow antimicrobial spectrum, low expression amount, poor solubility. In addition, the combination of the catalytic domain and the binding domain are not in a best condition, or there are other non-essential domains between them. Wild-type LysK includes two catalytic domains (CHAP and Amidase structural domains) while the Amidase structural domain has very low catalytic activity. When the Amidase structural domain is removed from the LysK (LysKΔ221-390), the activity of a protein is not changed (Becker S C, Dong S, Baker J R, et al. LysK CHAP endopeptidase domain is required for lysis of live staphylococcal cells [J]. FEMS microbiology letters, 2009, 294(1): 52-60.). An isolated CHAP catalytic domain of the LysK also has no antimicrobial activity in a TSB culture medium. In a complex environment, the catalytic domain needs to be combined with a binding domain to achieve more effect. However, the binding domain of the LysK has no great effect, and is leading to low affinity efficiency and narrow identification range. Therefore, construction of a highly active hybrid antimicrobial protein has great significance for promoting research and development of novel antimicrobial drugs. It will provide an effective solution for solving the problem of the bacteria of drug resistance.

In 2013, Gladskin, as the first bacteriophage lysin based antimicrobial protein product on the market in the world, is used for treatment of inflammatory skin diseases, such as eczema, rosacea and acne, caused by methicillin-resistant Staphylococcus aureus (MRSA) in intact skin. At present, clinical researches have already been conducted on many antimicrobial proteins, such as Lysostaphin (China, used for treatment of a wound infection with the Staphylococcus aureus), P128 (India, used for removal of the Staphylococcus aureus fixed to the nasal mucosa), CF301 (the United States of America, used for treatment of septicemia caused by the Staphylococcus aureus) and SAL200 (South Korea, used for treatment of the septicemia caused by the Staphylococcus aureus). In addition, clinical researches have also been conducted on some antimicrobial proteins, such as Cpl-1, ClyS, ClyH and PlyV12, which are used for treatment of infections caused by Staphylococcus aureus, Streptococcus pneumoniae and Enterococcus, respectively. All these antimicrobial proteins with clinical researches are focused on treatment of infections with G+ bacteria, and at present, there are no clinical researches on antimicrobial proteins used for treatment of G″ bacteria. Based on the severity of the drug-resistant G″ bacteria and the imbalance in development of antimicrobial protein drugs, it is urgent to develop new antimicrobial drugs against the G″ bacteria.

### SUMMARY OF THE INVENTION

An objective of the present invention is to provide a hybrid antimicrobial protein having a strong bactericidal effect and an application thereof, so as to overcome the defects of the prior art and meet human needs.

The hybrid antimicrobial protein of the present invention, having a strong bactericidal effect against Gram-negative bacteria, is a hybrid protein with the amino acid sequence of SEQ ID NO: 1 which named AB469; or a hybrid protein including a sequence similar to a catalytic domain of the AB469, or a hybrid protein including a sequence similar to a binding domain of the AB469; or, a hybrid protein including the catalytic and binding domains with positions interchanged of the AB469.

The AB469 includes 285 amino acids and has a molecular weight of 31.8 kD and a theoretical isoelectric point of 9.8. After purification in 2 steps, the purity can reach 95% or above. According to SDS-PAGE electrophoresis, it is shown that the AB469 has a molecular weight of about 32 kD.

A nucleic acid sequence encoding the AB469 is shown in SEQ ID NO: 2 and named ab469.

According to a test, it is proven that the AB469 has high solubility and stability.

According to an in vitro test, it is proven that the AB469 has high activity, a broad spectrum and a strong bactericidal effect on Acinetobacter baumannii, Pseudomonas aeruginosa, Klebsiella pneumoniae, Salmonella typhi and Escherichia coli. After adding 10 μg/mL of the AB469 into bacterial fluid and acts for 2 h, the Acinetobacter baumannii (ATCC 19606), the Pseudomonas aeruginosa (ATCC 15442) and the Salmonella typhi (CMCC 50094) can be decreased by more than 7 log values, and the Klebsiella pneumoniae (ATCC 700603) and the Escherichia coli (ATCC 35218) are decreased by more than 2 log values. After adding 50 μg/mL of the AB469 acts for 2 h, the Klebsiella pneumoniae (ATCC 700603) and the Escherichia coli (ATCC 35218) can be decreased by more than 7 log values, and Enterobacter cloacae (ATCC 700323) and Staphylococcus aureus (ATCC 6538) are decreased by about 4 log values.

The antimicrobial protein AB469 can effectively kill the clinically isolated Acinetobacter baumannii, the Pseudomonas aeruginosa and the Klebsiella pneumoniae. In addition, the AB469 has the same effect on the Acinetobacter baumannii, the Pseudomonas aeruginosa and the Klebsiella pneumoniae with multi-drug resistance (to ceftazidime, ceftriaxone, meropenem, imipenem, tobramycin and sulbactam).

The AB469 also has high activity under complex conditions such as serum and body fluid.

The hybrid protein consisting of a sequence similar to a catalytic domain of the AB469 also has high activity and is shown in SEQ ID NO: 3 and named AB46M9.

A nucleic acid sequence encoding the AB46M9 is shown in SEQ ID NO: 4 and named ab46m9.

The hybrid protein consisting of a sequence similar to a binding domain of the AB469 also has high activity and is shown in SEQ ID NO: 5 and named AB469M.

A nucleic acid sequence encoding the AB469M is shown in SEQ ID NO: 6 and named ab469m.

The hybrid protein consisting of the catalytic and binding domains with positions interchanged of the AB469, also has high activity and is shown in SEQ ID NO: 7 and named B946.

A nucleic acid sequence encoding the B946 is shown in SEQ ID NO: 8 and named b946.

Compared with wild-type ABgp46, the AB469 has one more binding domain, the antimicrobial activity in a complex environment can be significantly improved, and the complex environment includes certain ionic strength, a culture medium, serum and a body fluid.

According to a test, it is shown that under certain ionic strength (150 mM NaCl), the bactericidal activity of the ABgp46 is significantly reduced (Table 1), and a log decrease value is decreased from 5.58 to 1.54. The results are consistent with those of many proteins with only one catalytic domain (Osipovitch D C, Griswold K E. Fusion with a cell wall binding domain renders autolysin LytM a potent anti-Staphylococcus aureus agent [J]. FEMS microbiology letters, 2015, 362(2): 1-7.). From Table 1, it can be seen that the effect of the ionic strength on the ABgp46 is significantly higher than that on the AB469. A bactericidal effect of Oliverira-expressed ABgp46 (2 μM, ˜40 μg/mL) on the Pseudomonas aeruginosa (decreased by 4 log values) is lower than that on the Acinetobacter baumannii (decreased by more than 5 log values). The AB469 of the present invention with a lower protein concentration (0.31 μM, 10 μg/mL), has a strong bactericidal effect on the Acinetobacter baumannii and the Pseudomonas aeruginosa under 150 mM NaCl (decreased by more than 7 log values).

The ABgp46 is a bacteriophage lysin only including one catalytic domain and has been reported in detail in the following literature: Oliveira H, Boas D V, Mesnage S, et al. Structural and enzymatic characterization of ABgp46, a novel phage endolysin with broad anti-gram-negative bacterial activity [J]. Frontiers in microbiology, 2016, 7.

The hybrid antimicrobial protein having a strong bactericidal effect can be used for preparing an antimicrobial preparation. The antimicrobial preparation includes an antimicrobial effective amount of the hybrid antimicrobial protein having a strong bactericidal effect on Gram-negative bacteria and a drug carrier. The drug carrier includes an excipient, such as water and hydroxypropyl methyl cellulose.

Preferably, in the antimicrobial preparation, the hybrid antimicrobial protein having a strong bactericidal effect on Gram-negative bacteria has a weight percentage of 0.0001-0.05%.

The antimicrobial preparation can be applied to a surface of a human body or an object in need by spraying.

The present invention has the following beneficial effects.

In the present invention, on the basis of a large number of researches, a new hybrid antimicrobial protein is successfully constructed. The Escherichia coli or yeast is used for expression, and after purification, the hybrid antimicrobial protein with high purity is obtained. The protein can rapidly lyse the Gram-negative bacteria, such as the Acinetobacter baumannii, the Pseudomonas aeruginosa, the Klebsiella pneumoniae, the Salmonella typhi and the Escherichia coli. The antimicrobial preparation prepared from the antimicrobial protein can be used for prevention and treatment of bacterial infection. The antimicrobial protein is a new and efficient antimicrobial substance that can directly lyse bacteria. The antimicrobial protein can be prepared into an antimicrobial preparation used for prevention and treatment of bacterial infection and sterilization of medical apparatus and instruments and medical places.

### BRIEF DESCRIPTION OF DRAWINGS

FIG. 1 is an SDS-PAGE diagram of AB469, AB46M9, AB469M and B946.

FIG. 2 shows detection of biological activity of the AB469 against Acinetobacter baumannii (ATCC 19606) by using a turbidimetric method.

FIG. 3 shows detection of biological activity of the AB469 against Pseudomonas aeruginosa (ATCC 15442) by using a turbidimetric method.

FIG. 4 shows detection of biological activity of the AB469 against Klebsiella pneumoniae (ATCC 700603) by using a turbidimetric method.

FIG. 5 shows detection of biological activity of the AB469, the AB46M9, the AB469M and the B946 against the Acinetobacter baumannii (ATCC 19606) by using a turbidimetric method.

### DETAILED DESCRIPTION

The present invention is further described below in conjunction with specific embodiments, but the protection scope of the present invention is not limited thereto;

In the present invention, through analysis of a structural domain of an antimicrobial protein, a hybrid protein having high antimicrobial activity against Gram-negative bacteria is artificially designed, and an amino acid sequence is shown in SEQ ID NO: 1. Secretion expression of the protein in Escherichia coli is achieved by using a molecular biology method. After purification, a protein with high purity is obtained for activity detection.

### Embodiment 1 Construction, Expression and Purification of an AB469 Engineering Bacterium

### 1. Construction of an AB469 Engineering Bacterium

According to the preference of a codon of Escherichia coli, a nucleic acid sequence ab469 (SEQ ID NO: 2) encoding the AB469 was artificially designed and synthesized. A NcoI restriction endonuclease site (italic part) was added into a 5′ end, and a nucleic acid sequence ct was introduced with g at the end of the restriction site to form a codon encoding an alanine. A HindIII restriction endonuclease site was added into a 3′ end (italic part). The sequence was as follows:

ccatggctatcctgaccaaagatggctttggcatcattcgcaacgaact
gtttggcggcaaactggatcagacacaggtggatgccattaattttatt
gttgaaaaagctaccgaatctgggttaagttatccggaagcggcctatc
tgttagcgacgatctatcatgaaacgggtctgccgagcggttatcgtac
catgcagccaatcaaagaagccggtagtgataattacctccgctctaaa
aaatattatccgtatatcggctatggctatgttcagctgacgtggaaag
aaaattatggtcgtattggtaaactgatcggcatcgacttgatcaaaaa
tccggaaaaagccttagaaccgctgattgcgattcagattgccatcaaa
ggtatgctgaatggttggtttacaggtgtgggctttcgtcgcaaacgtc
cagtgagtaaatataacaagcagcagtatattgctgcacgcaatatcat
taatggtaaagataaagcagaactgatcgcgaaatatgccatcatcttt
gaacgcgccctgcgttctctgggctccaatagtacctctaatagtagca
ccaattcaggctctaccggtaaagtgagtctgccgaatcgtgtgattcg
cgtgaccaaaccgattgttcatggtagcgatgtgctggcaattcagaaa
gcactgtcaagcctgtatttttatccggaaaaaggtgccaaagataatg
gttccgatagctattatggtccgaaaaccgccaatgccgttaaacgctt
tcagtctgttaatggcttagttgcagatggcgtgtatggcccgaaaacc
cgcgcagccattctgaaaaaactgtaagctt

In order to facilitate expression in the Escherichia coli and to form a soluble target protein, Cys in an SP10 binding domain was mutated into Ser. An artificially synthesized ab469 gene inserted to a vector pET28a(+) through being connected between the NcoI and HindIII sites to obtain a recombinant plasmid pET28a-ab469. The recombinant plasmid was transformed into competent cells of Escherichia coli BL21 (DE3) to obtain an engineering bacterium expressing the AB469.

### 2. Expression and Purification of AB469

A monoclonal engineering bacterium was inoculated into an LB culture solution (containing 30 mg/L of kanamycin) and subjected to shaking culture overnight at 30° C., then inoculated into the same LB culture solution with a ratio of 1%, and subjected to shaking culture at 30° C. until an optical density (wavelength 600 nm) was about 0.6. An IPTG with a final concentration of 0.05 mM was added to induce protein expression and continuously subjected to shaking culture at 30° C., induction was conducted for about 4 h, and a fermentation supernatant was collected by centrifugation.

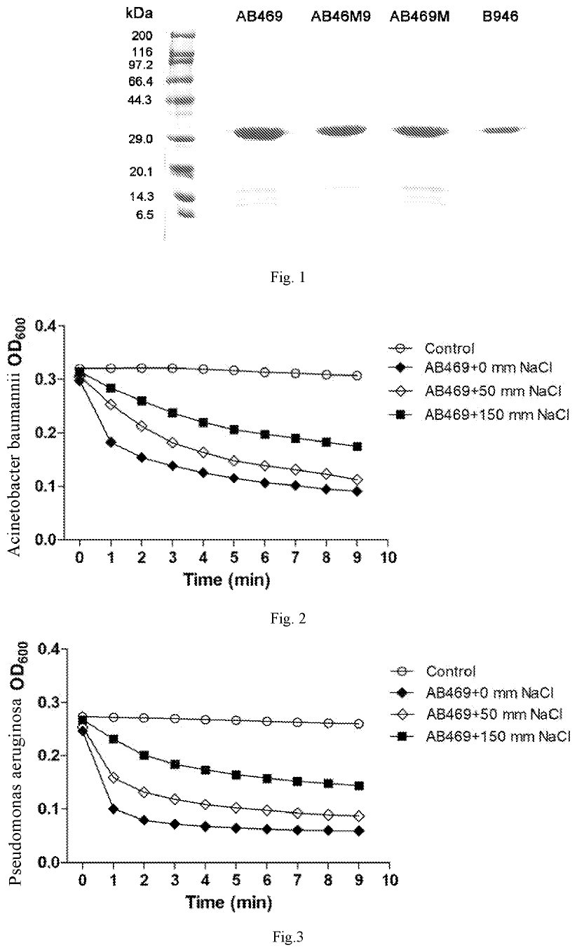

The fermentation supernatant was purified by two steps of cation exchange and gel filtration. A purified sample was cryopreserved at −20° ° C. for later use. The weight and purity of a recombinant protein sample were determined by using 10% SDS-PAGE. According to results, it could be seen that a main electrophoresis band of a recombinant hybrid antimicrobial protein AB469 was about 32 kD (see FIG. 1).

### Embodiment 2 Construction, Expression and Purification of Engineering Bacteria of Mutants AB46M9, AB469M and B946 of AB469

### 1. Construction of Engineering Bacteria of Mutants

The mutant AB46M9 (SEQ ID NO: 3) consisted of a catalytic domain AB46M (SEQ ID NO: 10) having 82% similarity with an amino acid sequence of AB46 (1-185aa) (SEQ ID NO: 9), a flexible linker, and a binding domain SP10 (154-236aa) (SEQ ID NO: 11), in sequence.

The mutant AB469M (SEQ ID NO: 5) consisted of a catalytic domain AB46 (1-185aa), a flexible linker, and a binding domain B9M (SEQ ID NO: 12) having 82% similarity with an amino acid sequence of SP10 (154-236aa), in sequence.

The B946 (SEQ ID NO: 7) consisted of a binding domain SP10 (154-236aa), a flexible linker in the middle, and a catalytic domain AB46 (1-185aa), in sequence.

According to the preference of a codon of Escherichia coli, nucleic acid sequences ab46m9 (SEQ ID NO: 4), ab469m (SEQ ID NO: 6) and b946 (SEQ ID NO: 8) that can encode AB46M9, AB469M and B946 were artificially designed and synthesized.

Construction of expression vectors and engineering bacteria of the ab46m9, the ab469m and the b946, and specific expression and purification methods of the ab46m9, the ab469m and the b946 were the same as those of the ab469. Purified AB46M9, AB469M and B946 samples were cryopreserved at −20° C. for later use. The weight and purity of recombinant protein samples were determined by using 10% SDS-PAGE (see FIG. 1).

### Embodiment 3 Detection of Bactericidal Activity of AB469 Against Acinetobacter baumannii, Pseudomonas Aeruginosa and Klebsiella pneumoniae by Using a Turbidimetric Method

The Acinetobacter baumannii (ATCC 19606), the Pseudomonas aeruginosa (ATCC 15442) and the Klebsiella pneumoniae (ATCC 700603) cultured overnight were transferred and allowed to grow to a mid-log phase (OD₆₀₀ was about 0.5). Bacterial bodies were collected by centrifugation (5,000 g, 5 min). The bacteria bodies were washed twice with 20 mM PBS (pH 7.2) and then resuspended in PBS buffers with different concentrations of NaCl (0-600 mM). A sample (to a final concentration of 2 μg/mL) or a PBS control, 10 μl of a 200 mM EDTA (to a final concentration of 0.5 mM) and 50 μl of a bacterial suspension were added into a 96-well plate, and the PBS was supplemented until a total reaction system was 200 μl. A reaction temperature was 37° C., and reading results was conducted at a wavelength of 600 nm (for 10 times at an interval of 1 min).

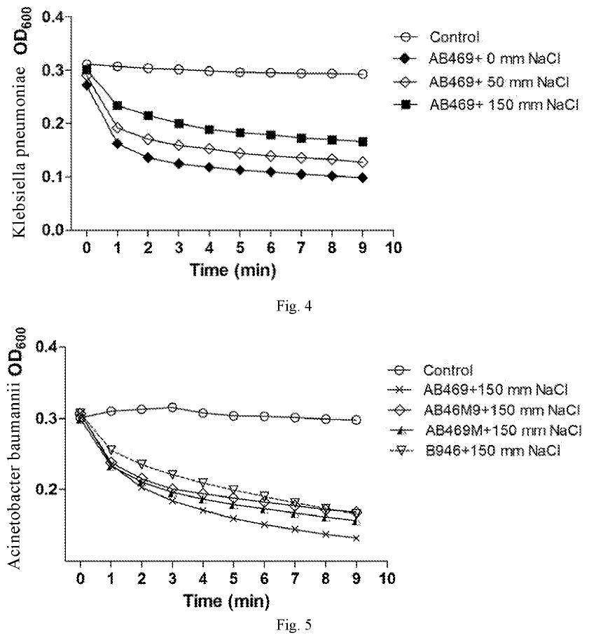

Turbidimetric detection results were shown in FIG. 2 to FIG. 4. The AB469 could lyse pathogenic bacteria, thereby causing the OD value of a bacterial solution decreased. When the OD value of the bacterial solution of the pathogenic bacteria was rapidly decreased, it was indicated that the AB469 had a rapid bactericidal effect on the Acinetobacter baumannii (ATCC 19606), the Pseudomonas aeruginosa (ATCC 15442) and the Klebsiella pneumoniae (ATCC 700603). With increase of the salt concentration, the AB469 still had high bactericidal activity. The bactericidal activity of the AB469 could be significantly improved by using some non-ionic surfactants (such as BriJ98, Tween 20 and Peregal).

### Embodiment 4 Detection of Bactericidal Activity of AB46M9, AB469M and B946 Against Acinetobacter baumannii by Using a Turbidimetric Method

The turbidimetric method was the same as that in Embodiment 3 for detection, and turbidimetric detection results were shown in FIG. 5. Compared with the AB469, the bactericidal activity of the mutants AB46M9, AB469M and B946 against the Acinetobacter baumannii was not significantly reduced.

### Embodiment 5 In Vitro Timed and Quantitative Killing Effects of AB469 on Standard Strains and Clinically Isolated Strains of Pathogenic Bacteria

Bacteria cultured overnight were transferred and allowed to grow to a mid-log phase (OD₆₀₀ was about 0.5). Bacterial bodies were collected by centrifugation (5,000 g, 5 min). The bacterial bodies were diluted with PBS (pH 7.2) to about 1*10⁸ cfu/mL. 1 mL of a sample containing the AB469 (to a final concentration of 10-50 μg/mL); NaCl (to a final concentration of 150 mM); and an EDTA (to a final concentration of 0.5 mM) or sodium citrate (to a final concentration of 8 mM) was added into 250 μl of a bacterial solution. After reaction at room temperature for 120 min, 0.5 mL of a sample solution was taken for serial dilution, and then 0.5 mL of a sample solution was taken and subjected to viable count culture. 40 uh/mL of AB46 and an EDTA (to a final concentration of 0.5 mM) were used as a control (after reaction at room temperature for 120 min). A preparation method of the AB46 was the same as that of the AB469.

According to results of an in vitro bactericidal experiment, it was shown that compared with the isolated AB46, the hybrid antimicrobial protein AB469 had stronger activity in killing Acinetobacter baumannii (ATCC 19606) (Table 1) and was more resistant to ionic strength.

TABLE 1
Comparison of bactericidal activity of ABgp46 and AB469
against Acinetobacter baumannii (ATCC 19606)

| | 0 mm NaCl | 150 mm NaCl |
|---|---|---|
| PBS/EDTA | 0.24 ± 0.12 | 0.16 ± 0.09 |
| ABgp46 (40 μg/mL)/EDTA | 5.58 ± 0.33 | 1.54 ± 0.23 |
| AB469 (10 μg/mL)/EDTA | >8.21 ± 0.57 | >7.8 ± 0.38 |

According to results of an in vitro bactericidal experiment (Table 2), it was shown that after 10 μg/mL of the AB469 acted for 2 h, the Acinetobacter baumannii (ATCC 19606), Pseudomonas aeruginosa (ATCC 15442) and Salmonella typhi (CMCC 50094) could be effectively killed and decreased by more than 7 log values; Klebsiella pneumoniae (ATCC 700603) and Escherichia coli (ATCC 35218) decreased by more than 2 log values; Enterobacter cloacae (ATCC 700323) and Staphylococcus aureus (ATCC 6538) decreased by about 1 log value.

According to results in Table 3, it was shown that after 50 μg/mL of the AB469 acted for 2 h, the Klebsiella pneumoniae (ATCC 700603) and the Escherichia coli (ATCC 35218) could be effectively killed; the Enterobacter cloacae (ATCC 700323) and the Staphylococcus aureus (ATCC 6538) decreased by about 4 log values.

TABLE 2
Bactericidal activity of AB469 (10 μg/mL)/EDTA against
different pathogenic bacteria under 150 mM NaCl

| | PBS/EDTA | AB469 (10 μg/mL)/EDTA |
|---|---|---|
| Acinetobacter baumannii | 0.16 ± 0.09 | >7.84 ± 0.38 |
| (ATCC 19606) | | |
| Pseudomonas aeruginosa | 0.56 ± 0.09 | >7.01 ± 0.27 |
| (ATCC 15442) | | |
| Salmonella typhi | 0.39 ± 0.14 | >7.59 ± 0.34 |
| (CMCC 50094) | | |
| Klebsiella pneumoniae | 0.27 ± 0.15 | 2.15 ± 0.18 |
| (ATCC 700603) | | |
| Escherichia coli | 0.26 ± 0.14 | 2.74 ± 0.13 |
| (ATCC 35218) | | |
| Enterobacter cloacae | 0.17 ± 0.06 | 0.90 ± 0.08 |
| (ATCC 700323) | | |
| Staphylococcus aureus | 0.22 ± 0.11 | 1.34 ± 0.16 |
| (ATCC 6538) | | |

TABLE 3
Bactericidal activity of AB469 (50 μg/mL)/EDTA against
different pathogenic bacteria under 150 mM NaCl

| | |
|---|---|
| PBS/EDTA | AB469 (50 μg/mL)/EDTA |

| | |
|---|---|
| Klebsiella pneumoniae | >7.36 ± 0.28 |
| (ATCC 700603) | |
| Escherichia coli | >7.62 ± 0.32 |
| (ATCC 35218) | |
| Enterobacter cloacae | 3.66 ± 0.25 |
| (ATCC 700323) | |
| Staphylococcus aureus | 4.58 ± 0.27 |
| (ATCC 6538) | |

After 10 μg/mL of the antimicrobial protein AB469 acted for 2 h, the clinically isolated Acinetobacter baumannii and Pseudomonas aeruginosa could be effectively killed (Table 4). The AB469 had the same effect on multi-drug (comprising ceftazidime, ceftriaxone, meropenem, imipenem, tobramycin and sulbactam, etc.) resistant strains. After 50 μg/mL of the antimicrobial protein AB469 acted for 2 h, the clinically isolated Klebsiella pneumoniae including multi-drug resistant bacteria could be effectively killed.

TABLE 4
Bactericidal activity of AB469/EDTA against different clinically
isolated pathogenic bacteria under 150 mM NaCl

| | Clinical | | | |
|---|---|---|---|---|
| Pathogenic | isolation | Drug | AB469 (10 | AB469 (50 |
| bacteria | number | resistance | μg/mL)/EDTA | μg/mL)/EDTA |
| Acinetobacter | 1512164 | − | >7.17 | |
| baumannii | 1511566 | − | >8.12 | |
| | 1512069 | − | >8.47 | |
| | 1511361 | + | >8.03 | |
| | 1511362 | + | >7.30 | |
| | 1511368 | + | >8.69 | |
| Pseudomonas | 1511387 | − | >7.75 | |
| aeruginosa | 1511410 | − | >7.65 | |
| | 1511475 | − | >7.92 | |
| | 1511386 | + | >6.71 | |
| | 1511389 | + | >7.47 | |
| | 1511433 | + | >7.30 | |
| Klebsiella | 1511549 | − | | >6.78 |
| pneumoniae | 1511691 | − | | >6.90 |
| | 1511674 | − | | >6.60 |
| | 1511673 | − | | >6.89 |
| | 1511558 | + | | >6.95 |
| | 1511535 | + | | >7.08 |

According to the results above, the hybrid AB469 had a very strong bactericidal effect on Gram-negative bacteria and the same effect on drug-resistant bacteria. The AB469 also had a strong bactericidal effect on the Staphylococcus aureus (Gram-positive bacteria).

FIG. 1 was provided.

### Embodiment 6

100 ml of a biological antimicrobial preparation containing AB469 was prepared.

A prescription was as follows:

| | | |
|---|---|---|
| AB469 | 0.02 | g |
| Anhydrous potassium dihydrogen phosphate | 0.16 | g |
| Anhydrous disodium hydrogen phosphate | 0.12 | g |
| Ethylenediaminetetraacetic acid disodium salt | 0.037 | g |
| Glycerol | 3 | ml |
| NaCl | 0.6 | g |

| | |
|---|---|
| Water | Supplemented |
| | to 100 ml |

A preparation method was as follows:
a) required amounts of auxiliary materials were calculated according to the prescription and the total volume of a preparation solution, accurately weighed and then put into a clean container;
b) the water that was 70% of the total preparation solution was added into a batching vessel to fully dissolve the NaCl, the ethylenediaminetetraacetic acid disodium salt, the disodium hydrogen phosphate and the potassium dihydrogen phosphate first, then the glycerol was added for uniform mixing, the AB469 was added, and finally, the water was added to reach a required volume for uniform mixing;
c) sterilization filtration: a prepared antimicrobial preparation was introduced into a sterilization filter, and a discharged antimicrobial preparation was put into a sterile container; and
d) filling: the sterile antimicrobial preparation was filled in a plastic or glass bottle.

The antimicrobial preparation could be used for sterilization and disinfection of a wound or a wound surface for 1-2 times a day.

1. A hybrid antimicrobial protein having a strong bactericidal effect, wherein the hybrid antimicrobial protein is: a hybrid protein named AB469 with an amino acid sequence shown in SEQ ID NO 1, and a hybrid protein comprising a sequence similar to a catalytic domain of the AB469, a hybrid protein comprising a sequence similar to a binding domain of the AB469, or a hybrid protein comprising the catalytic and binding domains with positions interchanged of the AB469.

2. A nucleic acid sequence, wherein the nucleic acid sequence encodes the AB469, shown in SEQ ID NO: 2 and named ab469.

3. The hybrid antimicrobial protein of claim 1, wherein, the hybrid protein comprising a sequence similar to a catalytic domain of the AB469 is shown in SEQ ID NO: 3 and named AB46M9.

4. A nucleic acid sequence encoding the AB46M9 of claim 3, shown in SEQ ID NO: 4 and named ab46m9.

5. The hybrid antimicrobial protein of claim 1, wherein, the hybrid protein comprising a sequence similar to a binding domain of the AB469 is shown in SEQ ID NO: 5 and named AB469M.

6. A nucleic acid sequence encoding the AB469M of claim 5, shown in SEQ ID NO: 6 and named ab469m.

7. The hybrid antimicrobial protein of claim 1, wherein, the hybrid protein comprising the catalytic and binding domains with positions interchanged of the AB469 is shown in SEQ ID NO: 7 and named B946.

8. A nucleic acid sequence encoding the B946 of claim 7, shown in SEQ ID NO: 8 and named b946.

9. An use of the hybrid antimicrobial protein of claim 1 in preparation of an antimicrobial preparation.

10. An antimicrobial preparation, comprising an antimicrobial effective amount of the hybrid antimicrobial protein of claim 1 and a drug carrier.

11. An use of the hybrid antimicrobial protein of claim 3 in preparation of an antimicrobial preparation.

12. An use of the hybrid antimicrobial protein of claim 5 in preparation of an antimicrobial preparation.

13. An use of the hybrid antimicrobial protein of claim 7 in preparation of an antimicrobial preparation.

14. An antimicrobial preparation, comprising an antimicrobial effective amount of the hybrid antimicrobial protein of claim 3 and a drug carrier.

15. An antimicrobial preparation, comprising an antimicrobial effective amount of the hybrid antimicrobial protein of claim 5 and a drug carrier.

16. An antimicrobial preparation, comprising an antimicrobial effective amount of the hybrid antimicrobial protein of claim 7 and a drug carrier.


SEQUENCE LISTING (12 sequences)

SEQ ID NO: 1 - protein, 285 aa, Artificial Sequence
  AB469
MAILTKDGFGIIRNELFGGKLDQTQVDAINFIVEKATESGLSYPEAAYLLATIYHETGLPSGYRTMQPIKEAGSDNYLRSKKYYPYIGYGYVQLTWKENYGRIGKLIGIDLIKNPEKALEPLIAIQIAIKGMLNGWFTGVGFRRKRPVSKYNKQQYIAARNIINGKDKAELIAKYAIIFERALRSLGSNSTSNSSTNSGSTGKVSLPNRVIRVTKPIVHGSDVLAIQKALSSLYFYPEKGAKDNGSDSYYGPKTANAVKRFQSVNGLVADGVYGPKTRAAILKKL

SEQ ID NO: 2 - DNA, 864 bp, Artificial Sequence
  ab469
ccatggctatcctgaccaaagatggctttggcatcattcgcaacgaactgtttggcggcaaactggatcagacacaggtggatgccattaattttattgttgaaaaagctaccgaatctgggttaagttatccggaagcggcctatctgttagcgacgatctatcatgaaacgggtctgccgagcggttatcgtaccatgcagccaatcaaagaagccggtagtgataattacctccgctctaaaaaatattatccgtatatcggctatggctatgttcagctgacgtggaaagaaaattatggtcgtattggtaaactgatcggcatcgacttgatcaaaaatccggaaaaagccttagaaccgctgattgcgattcagattgccatcaaaggtatgctgaatggttggtttacaggtgtgggctttcgtcgcaaacgtccagtgagtaaatataacaagcagcagtatattgctgcacgcaatatcattaatggtaaagataaagcagaactgatcgcgaaatatgccatcatctttgaacgcgccctgcgttctctgggctccaatagtacctctaatagtagcaccaattcaggctctaccggtaaagtgagtctgccgaatcgtgtgattcgcgtgaccaaaccgattgttcatggtagcgatgtgctggcaattcagaaagcactgtcaagcctgtatttttatccggaaaaaggtgccaaagataatggttccgatagctattatggtccgaaaaccgccaatgccgttaaacgctttcagtctgttaatggcttagttgcagatggcgtgtatggcccgaaaacccgcgcagccattctgaaaaaactgtaagctt

SEQ ID NO: 3 - protein, 285 aa, Artificial Sequence
  AB46M9
MALITKDGFSIIRNELFNGKLDQTQVDAINFLVEKSTEYGLTYPEAAYLLGTIYHETALPSAYRTMQPIKEAGSDSYLKSKKYYPYIGYGYVQLTWEDNYERIGKLIGIDLVKNPEKALEPLISIQIAIKGMLNGWFTAVRITRKRPVAKYNKQPYVAARNIINGKDRAELIPNYAFIFEKARRLIGSNSTSNSSTNSGSTGKVSLPNRVIRVTKPIVHGSDVLAIQKALSSLYFYPEKGAKDNGSDSYYGPKTANAVKRFQSVNGLVADGVYGPKTRAAILKKL

SEQ ID NO: 4 - DNA, 864 bp, Artificial Sequence
  ab46m9
ccatggctctgatcaccaaagatggctttagtatcattcgcaacgaactgtttaatggcaaactggatcagacacaggtggatgccattaattttctggttgaaaaatctaccgaatatgggttaacgtatccggaagcggcctatctgttaggtacgatctatcatgaaacggctctgccgagcgcttatcgtaccatgcagccaatcaaagaagccggtagtgatagttacctcaaatctaaaaaatattatccgtatatcggctatggctatgttcagctgacgtgggaagataattatgaacgtattggtaaactgatcggcatcgacttggttaaaaatccggaaaaagccttagaaccgctgattagtattcagattgccatcaaaggtatgctgaatggttggtttacagctgtgcgtatcacccgcaaacgtccagtggctaaatataacaagcagccgtatgttgctgcacgcaatatcattaatggtaaagatcgtgcagaactgatcccgaattatgcctttatctttgaaaaagcccgccgtctgatcggctccaatagtacctctaatagtagcaccaattcaggctctaccggtaaagtgagtctgccgaatcgtgtgattcgcgtgaccaaaccgattgttcatggtagcgatgtgctggcaattcagaaagcactgtcaagcctgtatttttatccggaaaaaggtgccaaagataatggttccgatagctattatggtccgaaaaccgccaatgccgttaaacgctttcagtctgttaatggcttagttgcagatggcgtgtatggcccgaaaacccgcgcagccattctgaaaaaactgtaagctt

SEQ ID NO: 5 - protein, 285 aa, Artificial Sequence
  AB469M
MAILTKDGFGIIRNELFGGKLDQTQVDAINFIVEKATESGLSYPEAAYLLATIYHETGLPSGYRTMQPIKEAGSDNYLRSKKYYPYIGYGYVQLTWKENYGRIGKLIGIDLIKNPEKALEPLIAIQIAIKGMLNGWFTGVGFRRKRPVSKYNKQQYIAARNIINGKDKAELIAKYAIIFERALRSLGSNSTSNSSTNSGSTGKVTLPSGVIRVTKPMVHGDDVLAIQKALAALYFYPDKGAKDNGIDGVYGPKTANAVKRFQSVNGLTADGIYGPKTRAAIEEKL

SEQ ID NO: 6 - DNA, 864 bp, Artificial Sequence
  ab469m
ccatggctatcctgaccaaagatggctttggcatcattcgcaacgaactgtttggcggcaaactggatcagacacaggtggatgccattaattttattgttgaaaaagctaccgaatctgggttaagttatccggaagcggcctatctgttagcgacgatctatcatgaaacgggtctgccgagcggttatcgtaccatgcagccaatcaaagaagccggtagtgataattacctccgctctaaaaaatattatccgtatatcggctatggctatgttcagctgacgtggaaagaaaattatggtcgtattggtaaactgatcggcatcgacttgatcaaaaatccggaaaaagccttagaaccgctgattgcgattcagattgccatcaaaggtatgctgaatggttggtttacaggtgtgggctttcgtcgcaaacgtccagtgagtaaatataacaagcagcagtatattgctgcacgcaatatcattaatggtaaagataaagcagaactgatcgcgaaatatgccatcatctttgaacgcgccctgcgttctctgggctccaatagtacctctaatagtagcaccaattcaggctctaccggtaaagtgacactgccgagtggtgtgattcgcgtgaccaaaccgatggttcatggtgacgatgtgctggcaattcagaaagcactggcagccctgtatttttatccggataaaggtgccaaagataatggtattgatggtgtttatggtccgaaaaccgccaatgccgttaaacgctttcagtctgttaatggcttaacagcagatggcatttatggcccgaaaacccgcgcagccattgaagaaaaactgtaagctt

SEQ ID NO: 7 - protein, 285 aa, Artificial Sequence
  B946
MAKVSLPNRVIRVTKPIVHGSDVLAIQKALSSLYFYPEKGAKDNGSDSYYGPKTANAVKRFQSVNGLVADGVYGPKTRAAILKKLGSNSTSNSSTNSGSTGILTKDGFGIIRNELFGGKLDQTQVDAINFIVEKATESGLSYPEAAYLLATIYHETGLPSGYRTMQPIKEAGSDNYLRSKKYYPYIGYGYVQLTWKENYGRIGKLIGIDLIKNPEKALEPLIAIQIAIKGMLNGWFTGVGFRRKRPVSKYNKQQYIAARNIINGKDKAELIAKYAIIFERALRSL

SEQ ID NO: 8 - DNA, 864 bp, Artificial Sequence
  b946
ccatggctaaagtgagtctgccgaatcgtgtgattcgcgtgaccaaaccgattgttcatggtagcgatgtgctggcaattcagaaagcactgtcaagcctgtatttttatccggaaaaaggtgccaaagataatggttccgatagctattatggtccgaaaaccgccaatgccgttaaacgctttcagtctgttaatggcttagttgcagatggcgtgtatggcccgaaaacccgcgcagccattctgaaaaaactgggctccaatagtacctctaatagtagcaccaattcaggctctaccggtatcctgaccaaagatggctttggcatcattcgcaacgaactgtttggcggcaaactggatcagacacaggtggatgccattaattttattgttgaaaaagctaccgaatctgggttaagttatccggaagcggcctatctgttagcgacgatctatcatgaaacgggtctgccgagcggttatcgtaccatgcagccaatcaaagaagccggtagtgataattacctccgctctaaaaaatattatccgtatatcggctatggctatgttcagctgacgtggaaagaaaattatggtcgtattggtaaactgatcggcatcgacttgatcaaaaatccggaaaaagccttagaaccgctgattgcgattcagattgccatcaaaggtatgctgaatggttggtttacaggtgtgggctttcgtcgcaaacgtccagtgagtaaatataacaagcagcagtatattgctgcacgcaatatcattaatggtaaagataaagcagaactgatcgcgaaatatgccatcatctttgaacgcgccctgcgttctctgtaagctt

SEQ ID NO: 9 - protein, 186 aa, Artificial Sequence
  AB46 1-185 aa
MAILTKDGFGIIRNELFGGKLDQTQVDAINFIVEKATESGLSYPEAAYLLATIYHETGLPSGYRTMQPIKEAGSDNYLRSKKYYPYIGYGYVQLTWKENYGRIGKLIGIDLIKNPEKALEPLIAIQIAIKGMLNGWFTGVGFRRKRPVSKYNKQQYIAARNIINGKDKAELIAKYAIIFERALRSL

SEQ ID NO: 10 - protein, 185 aa, Artificial Sequence
  AB46M
MLITKDGFSIIRNELFNGKLDQTQVDAINFLVEKSTEYGLTYPEAAYLLGTIYHETALPSAYRTMQPIKEAGSDSYLKSKKYYPYIGYGYVQLTWEDNYERIGKLIGIDLVKNPEKALEPLISIQIAIKGMLNGWFTAVRITRKRPVAKYNKQPYVAARNIINGKDRAELIPNYAFIFEKARRLI

SEQ ID NO: 11 - protein, 83 aa, Artificial Sequence
  SP10 154-236 aa
KVSLPNRVIRVTKPIVHGSDVLAIQKALSSLYFYPEKGAKDNGSDSYYGPKTANAVKRFQSVNGLVADGVYGPKTRAAILKKL

SEQ ID NO: 12 - protein, 83 aa, Artificial Sequence
  B9M
KVTLPSGVIRVTKPMVHGDDVLAIQKALAALYFYPDKGAKDNGIDGVYGPKTANAVKRFQSVNGLTADGIYGPKTRAAIEEKL